(19) United States
(12) Patent Application Publication
(10) Pub. No.: US 2026/0240598 A1
(43) Pub. Date: Aug. 20, 2026

(54) Methods of Diode Laser Treatment of Nasal Valve Region

(71) Applicant: Valent Medical Inc., Ridgefield, CT (US)

(72) Inventors: Yosef Krespi, Ridgefield, CT (US); Ron Hadani, Englewood, NJ (US)

(21) Appl. No.: 19/641,982

(22) Filed: Apr. 8, 2026

Related U.S. Application Data
(63) Continuation-in-part of application No. 18/938,113, filed on Nov. 5, 2024.

(51) Int. Cl. A61B 18/22; A61B 18/00

(52) U.S. Cl. CPC A61B 18/22; A61B 2018/00327; A61B 2018/00577

(57) ABSTRACT

There is provided a method for laser lipolysis of nasal valve obstruction which inserts a handheld laser device into a nostril of a patient. The device has a handpiece section and is connected to a diode laser at a proximal end of the device, with laser energy delivered along fiber optic internal to the handheld laser device inside the handpiece section. The fiber optic extends from a distal end of the handpiece section and is contained within a malleable sheath, culminating in a laser fiber optic tip at the distal end of the handheld laser device. The method delivers laser energy by the laser fiber optic tip to a selected treatment location of fibro-fatty tissue within a nasal valve of the nostril and ablates with laser energy any obstructing fibro-fatty tissue there. Treatments in tissue contact, non-contact, and interstitial modes are provided.

### CROSS-REFERENCE TO RELATED APPLICATIONS

This application claims benefit of and priority to U.S. application Ser. No. 18/938,113 filed on Nov. 5, 2024 and incorporated herein by reference in its entirety.

### FIELD OF THE INVENTION

The present invention relates to selective, targeted treatment of soft and hard tissues for the front of the nose and nasal vestibular area, and more particularly treatment of the nasal region with laser energy.

### BACKGROUND

With respect to the internal nasal valve, the internal nasal valve is the narrowest portion of the nasal airway passage and regulates airflow resistance. It is bounded medially by the nasal septum and tuberculum of Zuckerkandl, superiorly and laterally by caudal margin of ULC (upper lateral cartilage), its fibrofatty attachment to the pyriform aperture, and the anterior head of the inferior turbinate. The nasal valve angle typically ranges between 10° and 15°. The entire nasal valve surface area averages 55-64 mm². The patency of the internal nasal cavity is tested by Cottle's test, by stretching the nostrils laterally in order to demonstrate an improved airflow.

For physiology of the nasal valve, the dynamic of airflow follows a parabolic curve as it passes through the nasal valve. It contributes to air turbulence. This optimizes contact between the air stream and mucosal surface in order to obtain maximum air conditioning effect (humidification, filtration, olfaction and temperature control of inhaled air). The nasal valve should be considered as an inflow regulator. The main airflow resistance of the whole respiratory tract is normally confined to the nasal valve area.

For pathology, nasal valve problems can be primarily due to an inherent problem in this area, such as collapse of weaken or over resected alar cartilage during inspiration (i.e., result of esthetic rhinoplasty) or that the upper and lower lateral cartilages do not overlap. It is more common for this to occur secondary to septal deviation, turbinate hypertrophy or scarring of vestibular skin and this is termed secondary valve collapse.

Surgeons have treated the nasal valve region presently or in the past by: 1.) implants, such as with open rhinoplasty and cartilage reconstruction, and local stenting; 2.) surgical resection and 3.) non-surgical remodeling of nasal valve structures, such as with radio frequency (RF). These single repair methods only partially address anatomical problems of the valve region.

### SUMMARY OF THE INVENTION

The focus of the present invention is the laser induced changes to, and repair of the internal nasal valve region in order to improve the nasal airway patency in a unique fashion. The present invention is treating multiple sites and structures that compose the valve region, including erectile soft tissues and several cartilages. The present invention involves ablating, size reducing and shape changing of fatty tissue on one or more sides of the nostril to reduce obstructions and increase the cross-sectional area of the nasal valve section.

While the conditions of static obstruction in the nasal valve are common, no two nasal valves are the same, and the present invention targets a laser treatment of erectile soft tissue or cartilage as to improve the airway patency. The main areas targeted include soft tissues of the turbinate or swell bodies, septal cartilage, upper lateral cartilage, and lower lateral cartilage.

The present invention is a method for treatment of nasal valve obstructions where the method includes dilating and enlarging the nostril of a patient with a non-obstructing, self-retracting device to expose the nasal valve structures and then inserting a laser fiber tip which is connected to a laser handpiece into the nostril. The proximal end of the laser handpiece is connected to a diode laser source with a detachable trunk fiber optic cable. When connected to the proximal end of the laser handpiece, the distal end of the trunk fiber optic cable is connected internally to the proximal end of the laser fiber tip. The laser energy generated by the diode laser source is delivered along the trunk fiber optic cable to the laser fiber tip and to the treated nasal tissue. The fiber optic tip extends from a distal end of the laser handpiece and is contained within a malleable sheath where the fiber optic culminates as a fiber tip at the distal end of the laser handpiece. The method of the invention delivers laser energy by the fiber tip to a selected treatment location within the nasal valve section (nasal vestibule) and ablates obstructing tissue at the selected treatment location with the laser energy.

In different embodiments of the method of the present invention, the laser handpiece operates with the distal end of the laser fiber tip in relation to the treated tissue, in “non-contact mode”, elevating the tissue temperature to at 60 to 90 degrees Celsius to reshape soft cartilage, in “interstitial mode” for reducing volume of erectile structures such as turbinate or swell bodies and fibro-fatty tissue, or in “contact mode” shaving septal cartilage spur.

In embodiments of the present invention, the method of the present invention includes the selected treatment location known as the upper lateral cartilage and/or lower lateral cartilage. In an embodiment of the present invention, the method includes the selected treatment location as cartilaginous septal spurs. In an embodiment, the selected treatment location is fibro-fatty tissue. In an embodiment, the selected treatment location is a turbinate.

In another embodiment, the selected treatment location is swell bodies. In yet another embodiment, the selected treatment location is herniated soft tissue of a nasal floor, resembling swell bodies.

In an embodiment of the method, the present invention includes a laser handpiece which has a 300, 400, and up to 600 micron malleable laser fiber tip.

In an embodiment of the method, the present invention includes where the application of the laser energy for the treatment of nasal valve obstruction includes a continues mode, or pulse mode of the laser treatment.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below where:

FIG. 1 is an illustration of a side view of the external and internal nasal valve area.

FIG. 2A is an illustration of a bottom view of an anterior right nasal valve area.

FIG. 2B is an illustration of a bottom view of the right nasal valve area and angle.

FIG. 3A is an illustration of a bottom view of the common static obstructions in the left nasal valve area.

FIG. 3B is a table of the different obstructions in nasal valves.

FIG. 4 is an illustration of a nasal valve retractor.

FIG. 5 is an illustration of a nasal valve retractor in use.

FIG. 6 is a table which illustrates tissue reaction to different laser temperatures

FIG. 7 is an illustration of a fiber tip in use in a non-contact mode (no tissue contact).

FIG. 8A is an illustration of a diode laser device with a reusable handpiece for use in the present invention.

FIG. 8B is an illustration of a disposable handpiece for use with the diode laser device in the present invention.

FIG. 9 is an illustration of the handheld laser device in use on a patient.

FIG. 10 is an illustration of the tip of a laser device entering a patient's dilated nostril.

FIG. 11 is an illustration of the nasal valve locations of cavernous (erectile) regions.

FIG. 12 is an illustration of a nasal valve with fatty tissue deposits.

### DETAILED DESCRIPTION

The present invention is a device and method for laser treatment of nasal valve area (nasal vestibule), with improving visualization and access of a laser fiber tip to the treated area.

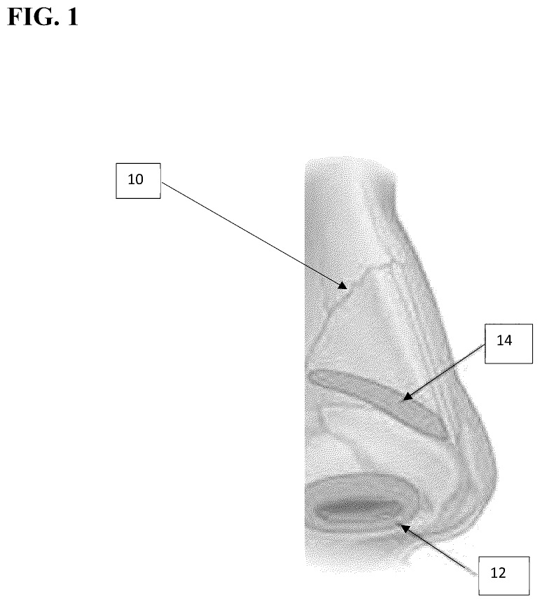

FIG. 1 is a side view of a nose 10 indicating the external nasal valve 12 and internal nasal valve 14. The external nasal valve is bordered at the superolateral by the caudal edge of upper lateral cartilage; on the lateral region nasal alar and bony piriform aperture of the maxilla; medial by the septum and columella. The integrity of the external nasal valve is tested by deep inspiration. The internal nasal valve regulates airflow resistance and is created at the junction of the upper lateral cartilage and the nasal septum. As stated above, the normal angle of the nasal valve is 10 to 15 degrees, and its patency is tested by Cottle's test.

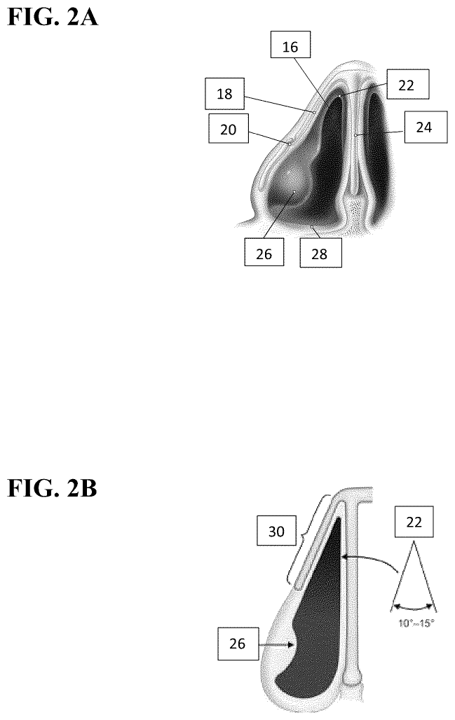

Referring to FIG. 2A and FIG. 2B, there is shown nasal valve area 16. As indicated on FIG. 2A, there is caudal edge upper lateral cartilage (ULC) 18, the scroll 20, the valve angle 22, the nasal septum 24 and head of inferior turbinate 26. The nasal floor 28 is indicated as well. In FIG. 2B, there is shown the nasal valve section 30 the head of the inferior turbinate 26 and the valve angle 22, indicated at 10 to 15 degrees.

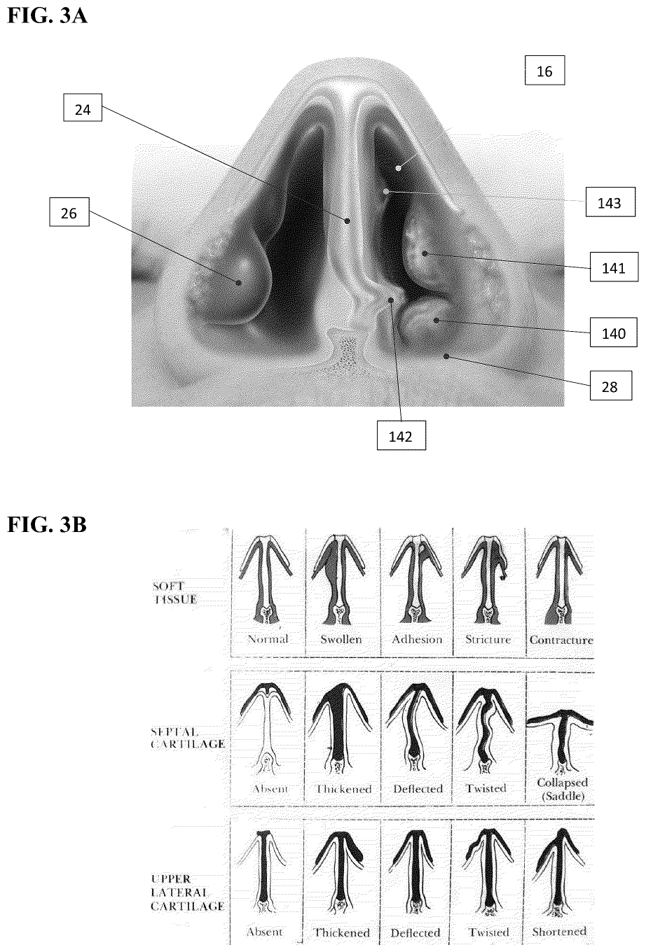

Referring to FIG. 3A, there is shown the nasal valve area of the left nostril 16, the nasal septum 24, with an illustration of the common static obstructions in a nasal valve. For example, septal swell body 143, hypertrophic inferior turbinate 141, vestibular swell body 140, and septal spur 142. These common static obstructions may come individually or in any combination. FIG. 3B is a table illustrating examples of static obstruction with the nasal valve. The top row of the table illustrates Soft Tissue type obstructions and shows, from left to right: normal tissue, then swollen, adhesion, stricture, and contracture. The middle row of the table illustrates Septal Cartilage type obstructions and shows first the absence of obstruction, then thickened, deflected, twisted, and finally collapsed (or saddle shaped). The lower row of the table has upper lateral cartilage (ULC) type obstruction and illustrates, from left to right, an absence of obstruction, thickened, deflected, twisted, and shortened obstructions.

As no two nasal valves are the same, the present invention targets the laser treatment site (erectile soft tissue or cartilage) to best improve the patency of the airway of the patient.

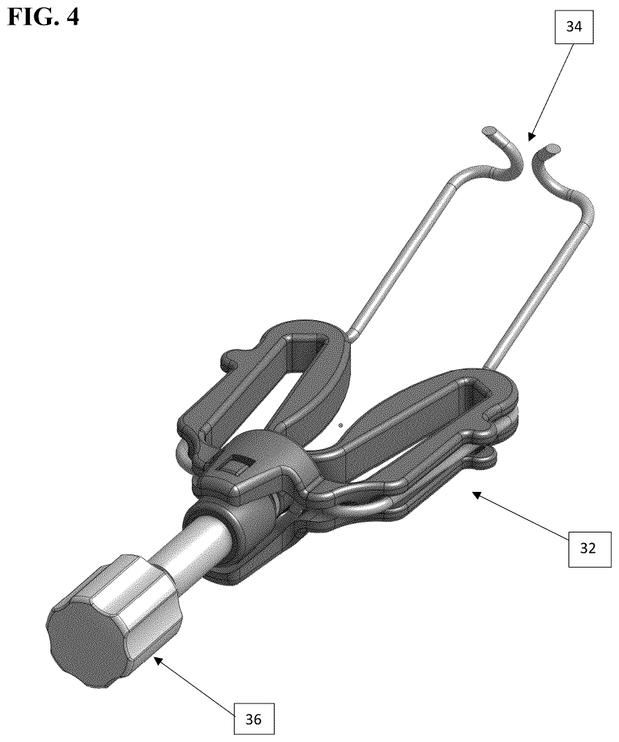

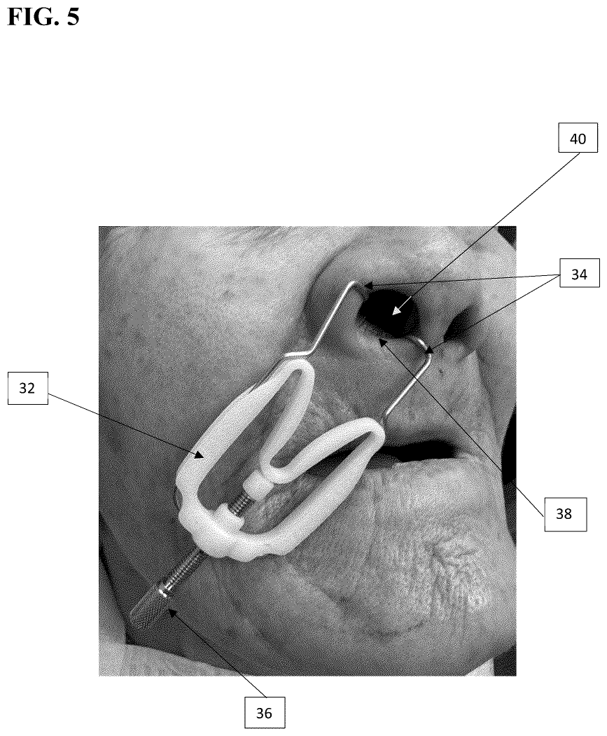

The present invention includes the improved exposure of the internal nasal valve using an adjustable and self-retaining nasal valve retractor. Referring to FIG. 4 and FIG. 5, there is shown a nasal valve retractor 32 and its use with a patient for the present invention. The nasal valve retractor 32 has a pair of distal end retracting prongs 34 which are controlled at the proximal end by an adjustment knob 36 or other similar mechanical adjustment means, such as clamps, handles, springs or tensions control mechanisms. By turning the adjustment knob 36, the medical professional is able to increase and decrease the distance between the distal prongs 34 during a procedure.

The retractor 32 is shown in FIG. 5 with the distal prongs 34 inserted into a nostril 38 of a patient. The retractor prongs 34 are adjusted in order to open up the nostril 38 and maintain the nostril 38 in a widened position so that the nasal cavity 40 is more exposed for the medical professional. A laser handpiece with its laser fiber tip is applied to the nostril and nasal valve area for ablating soft tissue and removing cartilaginous tissue for opening and improving airway patency. The present invention operates with the laser fiber tip being in contact with the treated tissue, non-contact with the treated tissue, or interstitially into the treated tissue of the selected nasal valve part which is undergoing the laser treatment.

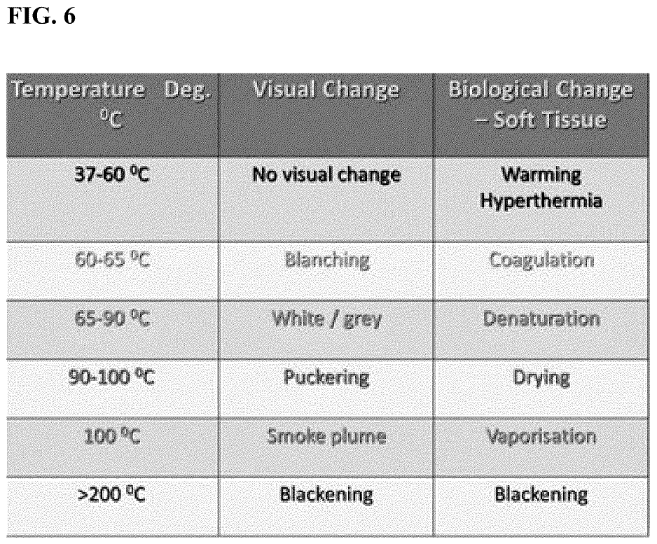

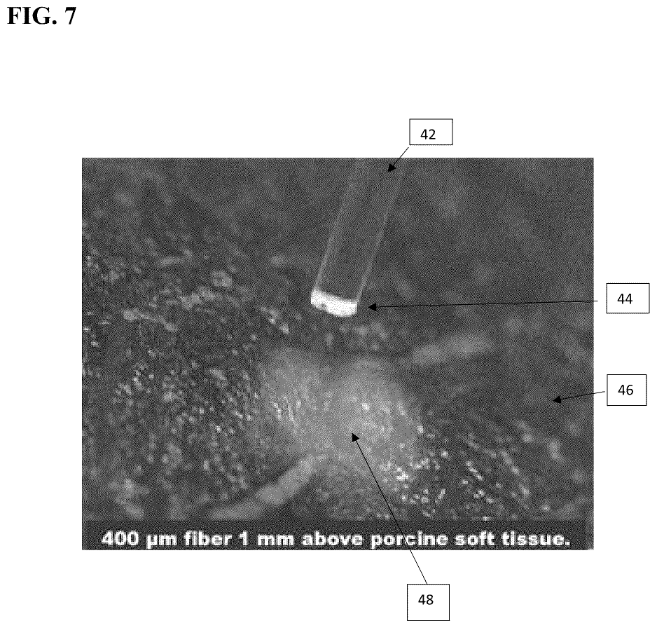

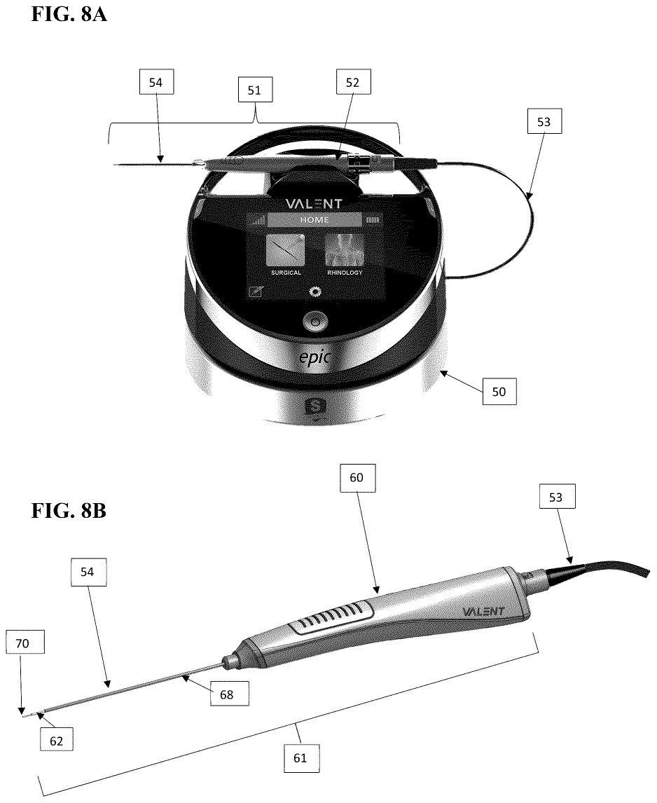

The reshaping of the ULC 18 (FIG. 2A) is performed in a non-contact mode where the distal tip of the laser fiber tip 70 (FIG. 8B) is at a distance of about 1 mm to 2 mm from the treated tissue. As described in FIG. 6 and FIG. 7, the non-contact mode provides the ability to visually monitor and control the temperature of the treatment of the ULC. In FIG. 6, the table indicates in the three columns: the temperature range, the visual change and the biological change for soft tissue. At 37-60 degrees Celsius, there is no visual change to the tissue and only warming hyperthermia. At 60 to 65 degrees Celsius, there is a visual change of blanching, and a biological tissue change of coagulation, and these are achieved in a non-contact mode. The ability of the medical professional to observe the blanching of the tissue provides an indication of the temperature of the treated tissue. At 65 to 90 degrees Celsius, there is a visual change of color to white/grey and a biological change of denaturation. The range of 60 to 90 degrees Celsius is an acceptable range for laser treatment in a non-contact mode.

At 90 degrees to 100 degrees Celsius, there is visual puckering of the tissue with drying biological change. At 100 degrees Celsius, there is smoke plumes and vaporization of the tissue. At temperatures of greater than 200 degrees Celsius, there is a visual blackening of the tissue and a corresponding blackening biological change. FIG. 7 illustrates a 400 μm laser fiber 42 at 1 mm above porcine soft tissue 46, used in the above test results. The tissue treatment area 48 is indicated below the fiber tip 44.

Referring to FIG. 8A and FIG. 8B, there is shown a diode laser 50 and a handheld laser device 51, and a disposable handheld laser device 61, which are used with the present invention for nasal valve treatment. The handheld laser device 51 is comprised of a reusable laser handpiece 52 and a disposable laser fiber tip 54. The diode laser 50 is attached to the reusable laser handpiece 52 as seen in FIG. 8A. In FIG. 8B, the disposable handheld laser device 61 is comprised of a disposable laser handpiece 60 and a disposable laser fiber tip 54. Each of the handheld laser device 51 and the disposable handheld laser device 61 are connected to the diode laser 50 with a laser trunk fiber 53, which is plugged into the proximal end of the reusable laser handpiece 52 or to the disposable laser handpiece 60. In the distal end of either of the laser handpieces 52 and 60 is the laser fiber tip 54 for performing treatment methods under this invention. The laser diode 50 together with the disposable handheld laser device 61 or with the handheld laser device 51 can perform three modes of ablation: non-contact, contact, and interstitial, which are used for treating all nasal valve regions, structures, and obstructions. The diode laser 50 has a power output of 0.5 to 10 Watts and is user selectable. The user can also select the diode laser to operate in either continuous wave or pulsed wave mode. The near infrared wavelength of the diode laser 50 (between 810 nm and 980 nm) provides for excellent coagulation, with no bleeding and no packing. The small diameter of 400 micron of the distal tip 70 of the laser fiber tip 54 provides pinpoint precision of energy-tissue application, which prevents collateral mucosa and tissue damage. The small profile of the laser fiber tip provides unobstructed direct tissue visualization, and when combined with the adjustable nasal valve retractor 32, amazing exposure of the nasal valve area is provided to the medical professional.

FIG. 8B. illustrates one embodiment of a disposable handheld laser device 61 for use with the method of the present invention. The disposable handheld laser device 61 connects to diode laser 50 with the laser trunk fiber 53, which is connected at the proximal end of the laser handpiece 60. The laser energy generated in the diode laser 50 is delivered along the laser trunk fiber 53. The distal end of the laser trunk fiber 53 is contacted with the proximal end of the laser fiber tip 54 inside the laser handpiece 60. The laser fiber tip 54 emits the laser energy from its distal end 70 to the treated tissue. Extending from the distal end of the laser handpieces 52 and 60, the laser fiber optic is contained within a hollow, malleable sheath 68 and culminates in a fiber tip 62 at the distal end 70. The fiber tip 62 delivers the laser energy to the selected treatment location.

There are numerous advantages of using the diode laser with a clear, malleable, and small diameter laser fiber tip. These include the providing the best wavelength (810 nm-980 nm) which is highly absorbed by hemoglobin (red). This results in high efficiency ablation of mucosal surface and excellent coagulation for prevention of bleeding. The malleability and small diameter allow the medical professional an easy access to hard-to-reach anatomies. It also results in unabstracted, direct visualization of the treated area, which allows the medical professional to visual monitoring of the tissue heating and complete control over the progress of the treatment. Visual monitoring can be achieved with rigid or flexible endoscope, miniaturized camera attached to the laser fiber tip, surgical microscope or simple headlight with surgical loops. The laser fiber tip 54 can be used in non-contact mode, raising the tissue temperature to about 60°-65° Celsius, selectively blanching and reshaping the mucosa effecting structures in sub-mucosa (i.e., cartilage). Delivering laser energy with the laser fiber tip 54 allows for minimal crusting and swelling. The laser fiber tip 54 also allows the medical professional a device and method of targeting all the regions around the entire nasal valve and provide effective ablation of mucosa, soft tissue, fat and cartilage.

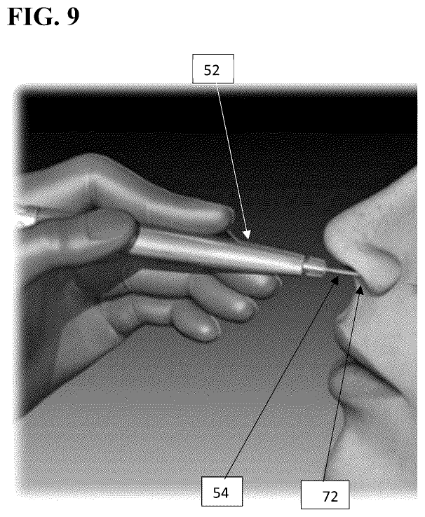

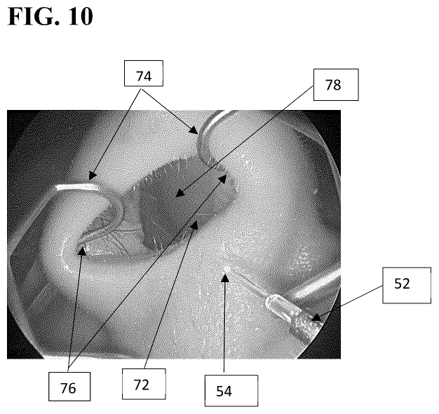

Referring to FIGS. 9 and 10, there is shown the reusable laser handpiece 52, (optionally a disposable laser handpiece 60) for the present invention in use by a medical professional. The laser handpiece 52 or 60 is inserted into the nostril of a patient 72 so that the medical professional can use the laser energy delivered by the laser fiber optic tip 54 for ablation of tissue. As seen in the view of the nostril 72 in FIG. 10, the retractor prongs 74 are placed inside the edges of the nostril wall 76 to dilate the nostril so that nasal cavity 78 can be more accessible. The medical professional can use the laser handpiece 52 or 60 so that the laser fiber tip 54 is inserted into the nasal cavity 78 as it is held open by the retractor. Ablating and treating tissue in any desired section of the nasal valve can be perform by the medical profession using the laser fiber tip 54 in either non-contact, interstitial or contact mode.

The methods of the present invention have numerous advantages. The diode laser is providing optimal energy-tissue application and is available in non-contact, contact, and interstitial ablation. The nasal valve treatment with diode laser of the present invention treats multiple valve conditions such as ULC (Upper Lateral Cartilage) and the LLC (Lower Lateral Cartilage), cartilaginous septal spurs, fibrofatty tissue, hypertrophic turbinate, swell bodies, soft tissue herniation nasal floor. The present invention with the 400 micron malleable tip enables full control and access to difficult to reach anatomy. This also allows for pinpoint precision treatment, prevents collateral damage, and results in less pain and faster patient recovery. The nasal valve treatment method incudes a pulse mode, as a unique feature allowing for tissue cooling and patient comfort. The laser energy emitted from the laser fiber tip creates an excellent coagulation with no bleeding and no packing of the treated area. The method of the present invention also allows for unobscured, direct tissue visualization of the treatment area.

The disposable, adjustable, self-retaining valve retractor provides better exposure of, and easier access to, the nasal valve region for treatment.

The diode laser can target and treat multiple nasal valve sites and conditions as indicated in the Table below:

| | | | Laser-Tissue | Power Level | | |
|---|---|---|---|---|---|---|
| | Site of Rx | Location | mode | {Watts] | When | Comments |
| 1 | Upper Lateral | Lateral | Non-contact | 3 W | Always | Single pass along ULC |
| 2 | Fibro Fatty Tissue | Lateral | Interstitial | 6 W | Always | 2 passes, towards pyriform aperture |
| 3 | Inferior Turbinate | Lateral | Interstitial | 6 W | Always | 2-3 passes |
| 4 | Septal Swell Body | Medial | Interstitial | 6 W | If needed | 2 passes |
| 5 | Septal Spur | Medial | Contact | 8 W | If needed | Cartilage shaving |
| 6 | Nasal Floor Fat | Base | Interstitial | 6 W | If needed | Single pass |

The present invention is a multi-site laser treatment based on three different laser-tissue application modes: non-contact; contact; and interstitial. The diode laser treatment utilizes these three different modes in treating the various conditions in the three locations (lateral, medial and base) of the nasal valve region.

Soft tissue ablation (volume reduction) is accomplished with “interstitial mode” by ablation of erectile (cavernous) structures of nasal valve such as the inferior turbinate; septal swell bodies; erectile nasal floor tissue.

Cartilage remodeling is accomplished with “non-contact mode”. This is used when treating the mucosa over the ULC (Upper Lateral Cartilage) and the LLC (Lower Lateral Cartilage). The direct and unobstructed real-time visualization of the treated area allows the medical professional to monitor and control the tissue temperature by observing the mucosa blanching and changing its color from red to pink to pale gray (60° C.-90° C.).

Contact mode of the present invention is used for cartilage shaving (i.e., cartilaginous septal spur, or deformed upper and lower lateral cartilages.

The method of ablation of lateral nasal fibro-fatty tissue occurs with “interstitial mode” of the laser fiber tip of the laser handpiece. Various combinations of the above modes are also in the scope of the present methods and invention.

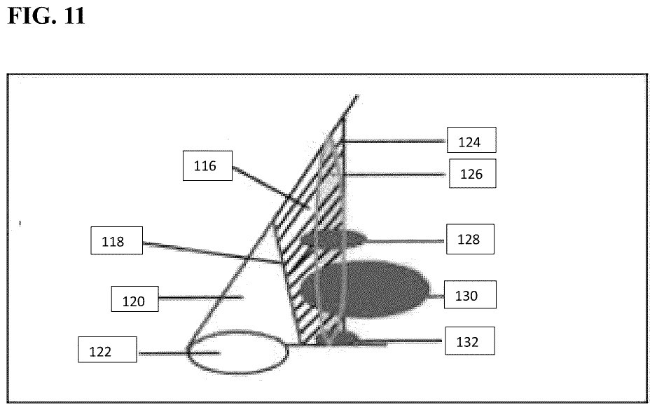

FIG. 11 indicates the location of erectile (cavernous) tissues of the nasal valve region. There is shown the nostril 122, the nasal vestibule 120, the ostrum internum 118 and the nasal valve 116. At the top of the nasal valve 116 is the isthmus nasi 124 and the pyriform orifice 126. The erectile structures the nasal septum cavernous body 128, the inferior nasal concha 130, and the erectile tissue of the nasal fossa floor 132.

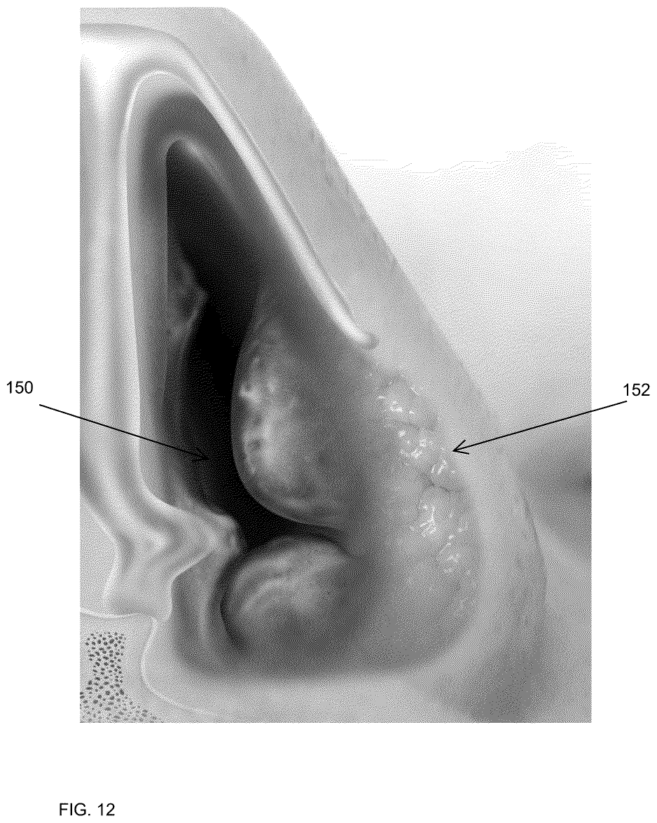

Referring to FIG. 12, there is shown an illustration of a nostril 150 which has a significant area of fibro-fatty tissue 152. The present invention is used for diode laser lipolysis of fibro-fatty tissues in the nasal valve region to improve nasal airflow. Laser lipolysis liquefies fatty tissue, coagulates small blood vessels, induces collagenesis (boosting collagen) with remodeling, and promotes tissue tightening. In use, the methods and devices described above are similarly used for diode laser lipolysis of the fibro-fatty tissue deposit to remove the fibro-fatty tissue by laser removal.

In practice for comprehensive management of the nasal valve region, a retrospective chart review involving 105 patients undergoing in-office laser treatment for management of nasal valve obstruction was performed under the Institutional Review Board (IRB). Eighty four (84) patients were found to have internal valve abnormalities (hypertrophy of inferior turbinates and/or the presence of septal spurs, and/or septal or vestibular swell bodies). Nineteen (19) patients were noted to also have external nasal valve dysfunction (nasal valve collapse, scarring) contributed by previous nasal surgeries such as rhinoplasty. A nasal endoscopy was used for diagnostic evaluation of nasal valve obstruction. Local anesthesia consisted of an injection of 2% lidocaine with epinephrine (1:100,000) to the inferior turbinate, and septal swell body and/or septal spur. The anterior aspect of the nasal valve region was further anesthetized with a sublabial injection. Laser treatment of the nasal valve was performed with a 940 nm diode laser, with 400-micron malleable fiber optic tip, used in three different modes: (1) contact, (2) non-contact, and (3) interstitial. Contact mode was used for addressing anterior cartilaginous septal spur, post rhinoplasty scars, synechiae, and deformities of the lower lateral cartilage. Non-contact mode involved blanching and reshaping the upper lateral cartilage. Interstitial mode was used to address turbinate hypertrophy and septal/vestibular swell bodies. The number of anatomical sites that were treated was dependent on the pathological conditions contributing to nasal airway obstruction. All patients underwent inferior turbinate reduction. Sixty (60) patients underwent septal swell body ablation (40 septal swell bodies only; 20 septal swell and vestibular swell bodies).

Twenty eight (28) patients underwent shaving of the septal spur. Seventeen (17) patients underwent upper lateral cartilage and fibrofatty tissue ablation (lipolysis). The procedures were performed bilaterally in 70% of the patients included in the study. Outcomes were assessed using pre- and post-procedure NOSE scores at 90 days post operatively.

The results indicated that there were no intra- or postoperative complications such as bleeding, crusting, scarring, or cosmetic changes. Patients demonstrated significant improvement in their NOSE scores 90 days post op. The NOSE scores were reduced from the baseline by 41 points (mean) in groups who underwent bilateral nasal valve treatment and by 45 points in patients who underwent unilateral nasal valve treatment.

This establishes that comprehensive, targeted laser therapy offers a personalized treatment for chronic nasal obstruction resulting from multiple anatomical abnormalities of the internal and external nasal valve regions. This office-based treatment modality is associated with minimal risk and pain and requires only local anesthesia. Improvement in NOSE scores 90 days post operatively suggests that laser therapy should be considered as preferred modality in the management of multi-site airway obstruction at nasal valve region.

As described above, tissue treatment around the external nasal valve is done with the present invention. The reasons tissue treatment may be necessary is usually due to: a.) lower lateral cartilage deformities, b.) scar formation post trauma, or c.) post rhinoplasty scar or adhesions. With the present invention, the surgeon can specifically use the nasal dilator for exposure and use the laser in contact mode for the external valve region.

With the 940 nm wavelength diode laser, and for the comprehensive treatment of nasal valve structures, a procedure starts with non-contact mode at 3-4 w, continuous wave (CW) to reshape and stiffen the upper lateral cartilage first, to continue with interstitial mode of ablation at 6-7 w, CW for ablating the fibrofatty tissues and erectile soft tissue structures of nasal valve region. In non-contact mode, the ability to raise the tissue temperature up to 65-70° C. and observing color changes of the mucosa endoscopically in real time is a feature to the use of this laser. In most patients, the non-contact mode can be performed using topical anesthetic gel (or pulsed wave instead of continuous wave if the patient is sensitive). Local anesthesia is used to treat the erectile soft tissue structures such as turbinates, swell bodies and vestibular fibrofatty tissues.

As described above herein, diode laser lipolysis is liquefying deep fibrofatty tissues around the nasal valve anterior to the head of the turbinate, which results in coagulation of small blood vessels, induces collagenases with tissue remodeling and eventually promoting tissue tightening at this critical area. Laser lipolysis is an established treatment modality in aesthetic procedures which boosts collagen formation and tightens many layers of the skin and subcutaneous (sub-Q). During the interstitial ablation, a cylindrical shape coagulation zone is created of approximately 2 mm in thickness, within fibrofatty tissues and erectile soft structures of the nasal valve region, which results in an improved, sustained open nasal airway.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for treatment of nasal valve obstruction by laser lipolysis comprising:
inserting a handheld laser device into a nostril of a patient;
said handheld laser device having a handpiece section and connected to a diode laser at a proximal end of said handheld laser device, with laser energy delivered along fiber optic internal to the handheld laser device inside said handpiece section; said fiber optic extending from a distal end of said handpiece section and contained within a malleable sheath; said fiber optic culminating in a laser fiber optic tip at the distal end of said handheld laser device;
delivering laser energy by said laser fiber optic tip to a selected treatment location of fibro-fatty tissue within a nasal valve of said nostril;
ablating obstructing fibro-fatty tissue at said selected treatment location of fibro-fatty tissue with said laser energy.

2. The method according to claim 1 further comprising as an initial step, the step of:
retracting a nostril of patient with a retracting device to expose a nasal valve.

3. The method according to claim 1 wherein said handheld laser device operates in contact mode at more than 100 degrees Celsius.

4. The method according to claim 1 wherein said handheld laser device operates in non-contact mode at 60 to 90 degrees Celsius.

5. The method according to claim 1 wherein said handheld laser device operates in interstitial mode at more than 100 degrees Celsius.

6. The method according to claim 1 wherein said selected treatment location is upper lateral cartilage.

7. The method according to claim 1 wherein said selected treatment location is lower lateral cartilage.

8. The method according to claim 1 wherein said selected treatment location is cartilaginous septal spurs.

9. The method according to claim 1 wherein said selected treatment location is a turbinate.

10. The method according to claim 1 wherein said selected treatment location is swell bodies.

11. The method according to claim 1 wherein said selected treatment location is herniated soft tissue of a nasal floor, like vestibular swell body.

12. The method according to claim 1 wherein said laser fiber optic tip is a 400 micron tip.

13. The method according to claim 1 wherein said laser fiber optic tip is a 400 micron malleable tip.

14. The method according to claim 1 wherein said treatment of nasal valve obstruction includes a pulse mode of laser treatment.

15. The method according to claim 1 wherein said treatment of nasal valve obstruction includes a continues mode of laser treatment.
